(19) United States
(12) Patent Application Publication
(10) Pub. No.: US 2011/0077410 A1
(43) Pub. Date: Mar. 31, 2011

(54) Process for the Preparation of 1-Phenyl-1,2,4-triazoles

(75) Inventors: Stefan ANTONS, Leverkusen (DE); Norbert Lui, Odenthal (DE)

(73) Assignee: Bayer CropScience AG, Monheim (DE)

(21) Appl. No.: 12/859,663

(22) Filed: Aug. 19, 2010

(30) Foreign Application Priority Data
Aug. 20, 2009 (EP) 09168286.4

(51) Int. Cl. C07D 249/08; C07C 251/28

(52) U.S. Cl. 548/269.2; 564/151

(57) ABSTRACT

The present invention relates to a process for the preparation of 1-phenyl-1,2,4-triazoles by converting phenyl hydrazides to N′-(iminomethyl)-N′-phenylacetyl hydrazides and the further reaction thereof by cyclization to give 1-phenyl-1,2,4-triazoles.

The present invention relates to a process for the preparation of 1-phenyl-1,2,4-triazoles by converting phenyl hydrazides to novel intermediates and the further reaction thereof by cyclization to give 1-phenyl-1,2,4-triazoles.

1-Phenyl-1,2,4-triazoles are generally known and valuable intermediates for the preparation of pest control compositions, in particular of insecticides (JP2007308485, EP1803712, WO2006128867, WO99/55668).

There is therefore a whole series of methods (e.g. Curtis, A. D. M Science of Synthesis, (2003), 13, 606) for preparing 1-phenyl-1,2,4-triazoles. In these, specific intermediates such as N-acylimines, N-acylamidines, N-formylamides, amidrazones or carbamoyl chlorides are passed through, which have to firstly be prepared, which is costly.

Journal für Praktische Chemie (Leipzig) (1988), 330 (3), 325-37 proposes the preparation of 1,2,4-triazoles from aryl-acylated phenyl hydrazides. The triazoles obtained in this way are isolated as perchlorate salts; these are very unacceptable for safety reasons.

Zhurnal Obshchei Khimii (1985), 55 (11), 2608-14 proposes to prepare the 1,2,4-triazoles starting from acylselenium compounds. Finally, the Journal of Heterocyclic Chemistry (1970), 7 (4), 821-9 describes the cyclization reaction of an acylphenylhydrazine with urea. However, this proposed method only produces a mixture of different cyclization products.

The examples indicated show that the current procedures are costly and/or require difficult-to-prepare or else very toxic feed materials. These processes are unsuitable for an industrial conversion.

The object of the present invention is therefore to provide a novel, simpler and industrially convertible process which produces the desired 1-phenyl-1,2,4-triazoles in good yield without having the disadvantages described above.

The object was achieved according to the present invention by a process for the preparation of 1-phenyl-1,2,4-triazoles of the general formula (I)

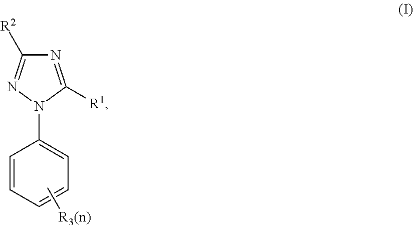

in which
R¹ is H, alkyl, haloalkyl, aryl, heteroaryl, alkylaryl, alkylheteroaryl,
R² is alkyl, haloalkyl, aryl, heteroaryl,
R³ (n) is n identical or different radicals selected from the group consisting of hydrogen, (C₁-C₅)-alkyl, (C₁-C₅)-haloalkyl, (C₁-C₅)-alkylthio, (C₁-C₅)-alkyl-SO—, (C₁-C₅)-alkyl-SO₂, (C₁-C₅)-haloalkylthio, (C₁-C₅)-haloalkyl-SO—, (C₁-C₅)-haloalkyl-SO₂, —SO₂-halogen, —SO₂Cl, —SO₂H, —SH; (C₁-C₅)-alkyl-O—, (C₁-C₅)-haloalkyl-O—, halogen, hydroxy, nitro, amino, (C₁-C₅)-alkylamino, (C₁-C₅)-bisalkylamino, halogen-(C₁-C₅)-alkoxy, aryl, heteroaryl, (C₁-C₅)-alkylaryl, (C₁-C₅)-alkylheteroaryl,
n is 1, 2, 3, 4, 5,
   characterized in that
   phenyl hydrazides of the general formula (II),

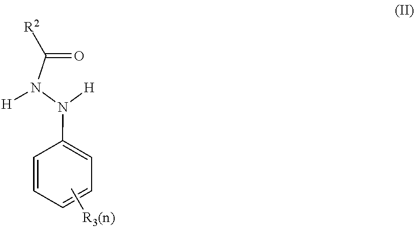

in which R², R³ and n have the meanings given above,
are converted to N′-(iminomethyl)-N′-phenylacetyl hydrazides of the general formula (III),

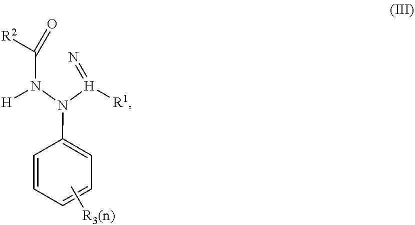

in which R¹, R², R³ and n have the meanings given above,
and these are cyclized with the elimination of water to give 1-phenyl-1,2,4-triazoles of the general formula (I).

This cyclization can take place with the assistance of water-withdrawing agents, such as, for example, orthoformic acid esters or acid chlorides or anhydrides. Also, the dewatering can take place by purely thermal means or with azeotropic removal of water. In this connection, solvents such as, for example, toluene or xylene can be used.

The process according to the invention can be illustrated by reference to the following scheme A

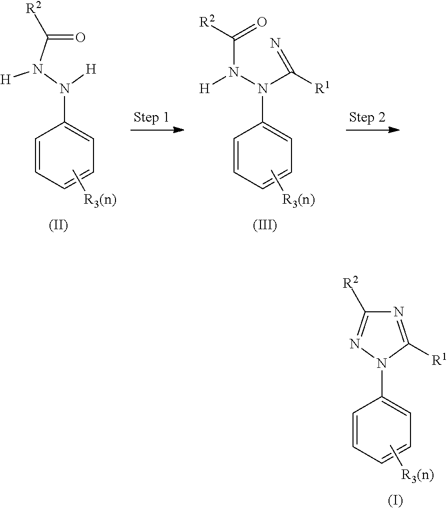

where R¹, R², R³ and n have the meanings given above.

### Scheme A

### General Definitions

Within the context of the present invention, the term “alkyl”, either on its own or in combination with further terms, such as, for example, haloalkyl, is understood as meaning a radical of a saturated, aliphatic hydrocarbon group having 1 to 12 carbon atoms which may be branched or unbranched. Examples of C₁-C₁₂-alkyl radicals are methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1-ethylpropyl, 1,2-dimethylpropyl, hexyl, n-heptyl, n-octyl, n-nonyl, n-decyl, n-undecyl and n-dodecyl. Of these alkyl radicals, C₁-C₆-alkyl radicals are particularly preferred. C₁-C₄-Alkyl radicals are especially preferred.

According to the invention, the term “aryl” is understood as meaning an aromatic radical having 6 to 14 carbon atoms, preferably phenyl.

The term “arylalkyl” is understood as meaning a combination of radicals “aryl” and “alkyl” defined according to the invention, where the radical is generally bonded via the alkyl group. Examples thereof are benzyl, phenylethyl or α-methylbenzyl, where benzyl is particularly preferred.

Within the context of the present invention, radicals substituted by halogen, for example haloalkyl, are understood as meaning radicals halogenated one or more times up to the maximum possible substituent number. In the case of multiple halogenation, the halogen atoms may be identical or different. Halogen here is fluorine, chlorine, bromine or iodine, in particular fluorine, chlorine or bromine.

The term “alkoxy”, either on its own or in combination with further terms, such as, for example, haloalkoxy, is in the present case understood as meaning a radical O-alkyl, where the term “alkyl” has the above meaning.

Optionally substituted radicals may be mono- or polysubstituted, where, in the case of a multiple substitution, the substituents may be identical or different.

### Phenyl Hydrazides of the Formula (II)

The phenyl hydrazides used as starting materials when carrying out the process according to the invention are generally defined by the formula (II)

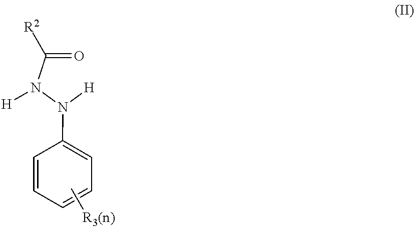

where R², R³ and n have the meanings given above.

They can be prepared by methods known in the literature. The aromatic hydrazines are reacted with carboxylic acid esters (Izvestiya Sibirskogo Otdeleniya Akademii Nauk SSSR, Seriya Khimicheskikh Nauk, (2), 120-4, 1980; Turkish Journal of Chemistry, 26 (2), 159-169, 2002), with acid anhydrides (WO 2008/134969, U.S. Pat. No. 5,010,068, 23 Apr. 1991) or else with acid halides (Farmaco, 49 (2), 97-104, 1994) to give the corresponding hydrazides of the general formula (II).

According to the invention, preference is given to the following hydrazides of the formula (II) as starting materials

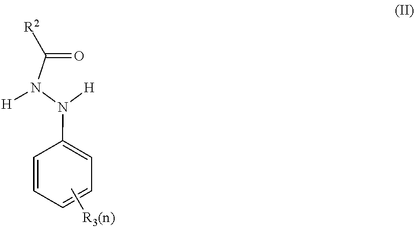

in which
R² is preferably alkyl, haloalkyl, aryl,
   is particularly preferably haloalkyl,
   is very particularly preferably CF₃, CHF₂ or CF₂CF₃ or CF₂CF₂Cl.
R³ (n) is preferably n identical or different radicals selected from the group consisting of (C₁-C₅)-alkyl, (C₁-C₅)-haloalkyl, halogen, nitro,
   is particularly preferably n identical or different radicals selected from the group consisting of (C₁-C₅)-alkyl, halogen.
n is preferably 1, 2, 3, 4, 5,
n is particularly preferably 1, 2, 3, 4.

### N′-(Iminomethyl)-N′-phenylacetyl hydrazide compounds of the formula (III)

The compounds formed as intermediates according to the present invention are generally defined by the formula (III)

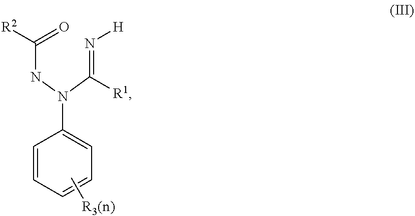

where
R¹ is H, alkyl, haloalkyl, aryl, heteroaryl, alkylaryl, alkylheteroaryl,
R¹ is preferably H, alkyl, haloalkyl, aryl,
R¹ is particularly preferably H or alkyl.
R² is alkyl, haloalkyl, aryl, heteroaryl,
R² is preferably alkyl, haloalkyl, aryl,
R² is particularly preferably haloalkyl,
R² is very particularly preferably CF₃, CHF₂, CF₂CF₃, CF₂CF₂Cl.
R³ (n) is n identical or different radicals selected from the group consisting of hydrogen, (C₁-C₅)-alkyl, (C₁-C₅)-haloalkyl, (C₁-C₅)-alkylthio, (C₁-C₅)-alkyl-SO—, (C₁-C₅)-alkyl-SO₂, (C₁-C₅)-haloalkylthio; (C₁-C₅)-haloalkyl-SO—, (C₁-C₅)-haloalkyl-SO₂—, SO₂-halogen, —SO₂Cl, —SO₂H, —SH; (C₁-C₅)-alkyl-O—, (C₁-C₅)-haloalkyl-O—, halogen, hydroxy, nitro, amino, (C₁-C₅)-alkylamino, (C₁-C₅)-bisalkylamino, halogen-(C₁-C₅)-alkoxy, aryl, heteroaryl, (C₁-C₅)-alkylaryl, (C₁-C₅)-alkylheteroaryl,
R³ (n) is preferably n identical or different radicals selected from the group consisting of (C₁-C₅)-alkyl, (C₁-C₅)-haloalkyl, halogen, nitro,
R³ (n) is particularly preferably n identical or different radicals selected from the group consisting of (C₁-C₅)-alkyl, halogen.
n is preferably 1, 2, 3, 4, 5,
n is particularly preferably 1, 2, 3, 4.

The preparation of the novel compounds of the formula (III) can take place in different ways.

Thus, e.g. the acylphenyl hydrazides of the formula (II) can be reacted with amidinium salts of the general formula (IV) to give compounds of the formula (III),

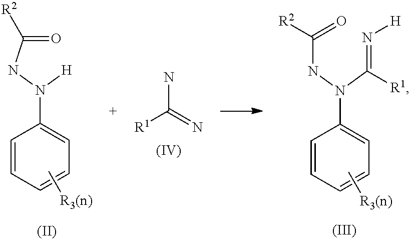

where R¹, R², R³ and n have the meanings given above.

Alternatively, the acylphenyl hydrazides of the formula (II) can be reacted with ammonium salts of the formula (VI) or else only with ammonia in the presence of formic acid derivatives of the formula (V) to give compounds of the formula (III),

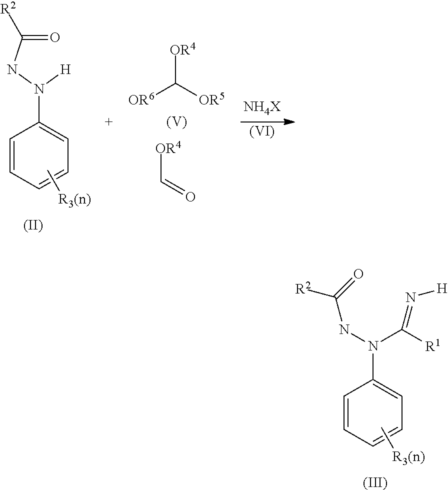

where
R¹, R², R³ and n have the meanings given above and
R⁴ is hydrogen, (C₁-C₅)-alkyl, (C₁-C₅)-haloalkyl, aryl, heteroaryl, (C₁-C₅)-alkylaryl, (C₁-C₅)-alkylheteroaryl,
R⁵ is hydrogen, (C₁-C₅)-alkyl, (C₁-C₅)-haloalkyl, aryl, heteroaryl, (C₁-C₅)-alkylaryl, (C₁-C₅)-alkylheteroaryl,
R⁶ is hydrogen, (C₁-C₅)-alkyl, (C₁-C₅)-haloalkyl, aryl, heteroaryl, (C₁-C₅)-alkylaryl, (C₁-C₅)-alkylheteroaryl.
X is an organic or inorganic acid radical R⁷COO—, Hal-, HSO₄—, SO₄—,
and R⁷ is hydrogen, (C₁-C₅)-alkyl, (C₁-C₅)-haloalkyl, aryl, heteroaryl, (C₁-C₅)-alkylaryl, (C₁-C₅)-alkylheteroaryl.

### Solvents

In general, it is advantageous to carry out the process according to the invention in the presence of solvents (diluents). Solvents are advantageously used in an amount such that the reaction mixture remains readily stirrable throughout the entire reduction process. Suitable solvents for carrying out the process according to the invention are all organic solvents that are inert under the reaction conditions.

Examples which can be mentioned are: halogenated hydrocarbons, in particular chlorinated hydrocarbons, such as tetrachloroethylene, tetrachloroethane, dichloropropane, methylene chloride, dichlorobutane, chloroform, tetrachloromethane, trichloroethane, trichloroethylene, pentachloroethane, difluorobenzene, 1,2-dichloroethane, chlorobenzene, bromobenzene, dichlorobenzene, chlorotoluene, trichlorobenzene; alcohols such as methanol, ethanol, isopropanol, butanol; ethers, such as ethyl propyl ether, methyl tert-butyl ether, n-butyl ether, anisole, phenetol, cyclohexyl methyl ether, dimethyl ether, diethyl ether, dimethyl glycol diphenyl ether, dipropyl ether, diisopropyl ether, di-n-butyl ether, diisobutyl ether, diisoamyl ether, ethylene glycol dimethyl ether, isopropyl ethyl ether, tetrahydrofuran, methyltetrahydrofuran, dioxane, dichlorodiethyl ether and polyethers of ethylene oxide and/or propylene oxide; amines such as trimethyl-, triethyl-, tripropyl-, tributylamine, N-methylmorpholine, pyridine, alkylated pyridines and tetramethylenediamine; aliphatic, cycloaliphatic or aromatic hydrocarbons such as pentane, n-hexane, n-heptane, n-octane, nonane and technical-grade hydrocarbons which may be substituted by fluorine and chlorine atoms, such as methylene chloride, dichloromethane, trichloromethane, tetrachloromethane, fluorobenzene, chlorobenzene or dichlorobenzene; for example so-called white spirits with components having boiling points in the range for example from 40° C. to 250° C., cymene, petroleum spirit fractions within a boiling interval from 70° C. to 190° C., cyclohexane, methylcyclohexane, petroleum ether, ligroin, octane, benzene, toluene, chlorobenzene, bromobenzene, nitrobenzene, xylene; esters such as methyl acetate, ethyl acetate, butyl acetate, isobutyl acetate, and also dimethyl carbonate, dibutyl carbonate, ethylene carbonate; and aliphatic alcohols, such as methanol, ethanol, n-propanol and isopropanol and n-butanol.

Particularly preferred solvents are nitriles such as e.g. acetonitrile or benzonitrile.

However, it is also possible for one of the reagents themselves to serve as solvent, such as e.g. triethyl orthoformate.

As a rule, the reaction is carried out at atmospheric pressure, although it can likewise be carried out at reduced pressure or, if required, even at increased pressure.

Preferred temperatures here are 20 to 200° C.

Based on the hydrazide used, 1 to 20 equivalents of ammonia or of an ammonium salt are used. Preference is given to 1 to 3 equivalents.

Based on the hydrazide used, 1 to 20 equivalents of the acid component of the general formula V are used. Preference is given to 3 to 10 equivalents.

If the triesters of orthoformic acid are used, primarily the (C₁-C₅)-alkyl esters are suitable, such as e.g. trimethyl orthoformate, triethyl orthoformate, tri-n-propyl orthoformate, triisopropyl orthoformate, tri-n-butyl orthoformate or triisobutyl orthoformate.

Since the alkoxy radicals are cleaved off as alcohols during the reaction, the reaction can take place with simultaneous removal by distillation of these alcohols and thus, if appropriate, be speeded up.

The reactants can be added together in various sequences. As a rule, the solids are introduced as initial charge and then the formate is added and the mixture is heated to the reaction temperature.

When conversion is complete, the product is isolated by customary methods such as e.g. crystallization and, in the case of relatively volatile products, distillation. Reaction media and reagents used in excess can be recovered using customary methods and be reused.

The formamidine may be in the form of a salt of an inorganic or organic acid, such as e.g. hydrochloric acid, carbonic acid or formic acid or acetic acid. The formamidine can then be reacted in the presence of an organic or inorganic base. Examples of such bases are NaOH, NaHCO₃, Na₂CO₃, K₂CO₃, KHCO₃ etc. or NEt₃, i-prop₂NEt, pyridine etc.

Instead of formamidine or ammonium salts, it is also possible to use only ammonia. This can be introduced in gaseous form or be added in the form of a solution.

### EXAMPLES

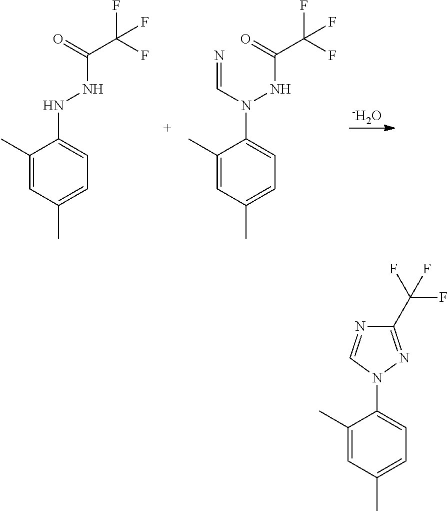

### Example 1

### Reaction of N′-(2,4-dimethylphenyl)-2,2,2-trifluoroacetohydrazide with ammonium acetate and ethyl orthoformate to give N′-(2,4-dimethylphenyl)-2,2,2-trifluoro-N′-(iminomethyl)acetohydrazide

5 g of N′-(2,4-dimethylphenyl)-2,2,2-trifluoroacetohydrazide are stirred together with 9.574 g of triethyl orthoformate and 4.15 g of ammonium formate at 20° C. The solid precipitated out overnight is filtered off and washed with a small amount of ortho ester. In this way, 4.6 g of 97% strength product are isolated as acetate salt. The mother liquor contains, besides further N′-(2,4-dimethylphenyl)-2,2,2-trifluoro-N′-(iminomethyl)acetohydrazide, already formed 1-(2,4-dimethylphenyl)-3-(trifluoromethyl)-1H-1,2,4-triazole and some starting material.

N′-(2,4-Dimethylphenyl)-2,2,2-trifluoro-N′-(iminomethyl)acetohydrazide: H-NMR (DMSO) 8.5-9 ppm (brs, 1H); 8.05 ppm (s, 1H); 7.5-7.8 ppm (br, 1H); 7.25 ppm (d, 1H); 7.08 ppm (s, 1H); 7.02 ppm (d, 1H); 2.26 ppm (s, 3H); 2.27 ppm (s; 3H), 1.9 ppm (s, 3H).

### Example 2

### Reaction of N′-(2,4-dimethylphenyl)-2,2,2-trifluoroacetohydrazide with formamidinium acetate and sodium hydrogen carbonate to give N′-(2,4-dimethylphenyl)-2,2,2-trifluoro-N′-(iminomethyl)acetohydrazide

5 g (0.09 mol) of N′-(2,4-dimethylphenyl)-2,2,2-trifluoroacetohydrazide are stirred together with 2.69 g of formamidinium acetate and 4.52 g of NaHCO3 in 30 ml of acetonitrile at 20° C. On the next day, the acetonitrile is distilled off under reduced pressure. The residue is admixed with water and extracted twice with methylene chloride. Distilling off the methylene chloride gives 5 g of a 17% strength product. In addition, 65% of 1-(2,4-dimethylphenyl)-3-(trifluoromethyl)-1H-1,2,4-triazole have already formed. The remainder of 18% is unreacted N′-(2,4-dimethylphenyl)-2,2,2-trifluoroacetohydrazide.

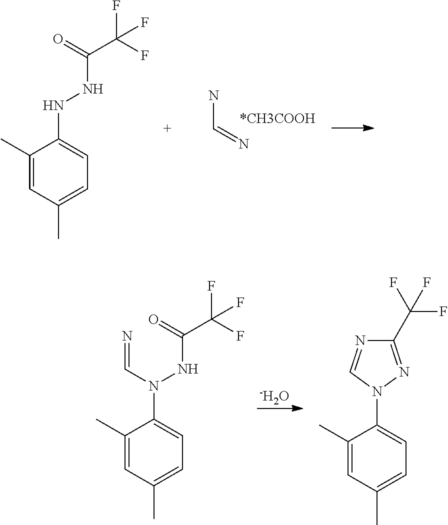

### Example 3

### Reaction of N′-(2,4-dimethylphenyl)-2,2,2-trifluoroacetohydrazide with formamidinium chloride and sodium hydrogencarbonate

5 g (0.1 mol) of N′-(2,4-dimethylphenyl)-2,2,2-trifluoroacetohydrazide are stirred together with 2.08 g of formamidinium hydrochloride and 4.522 g of sodium hydrogencarbonate in 30 ml of acetonitrile for 24 hours at RT. The inorganic salts are filtered off and washed with a small amount of acetonitrile. After removing the acetonitrile using a rotary evaporator, 5 g of a 24% strength crude product are obtained. The remainder consisted of already cyclized 1-(2,4-dimethylphenyl)-3-(trifluoromethyl)-1H-1,2,4-triazole.

1-(2,4-Dimethylphenyl)-3-(trifluoromethyl)-1H-1,2,4-triazole; H-NMR (CDCl3): 8.3 ppm (s, 1H); 7.1-7.25 (s+2d, 3H); 2.4 ppm (s, 3H); 2.2 ppm (s, 3H)

### Example 4

### Reaction of N′-(2,4-dimethylphenyl)-2,2-difluoroacetohydrazide with ammonium acetate and ethyl orthoformate to give N′-(2,4-dimethylphenyl)-2,2-difluoro-N′-(iminomethyl)acetohydrazide

| Reagent | Conditions | Yield | Remarks |
|---|---|---|---|
| HC(OMe)3 | 12 h/100° C. | 69.7% | |
| HC(Met)3 | 12 h/100° C. | 68% | |
| Toluene | 24 h/110° C. | 83% | Dewatering on the |
| | | | water separator |
| Without | 12 h/100° C. | 98% | The product was |
| | | | treated as a melt |

### Example 5a

### Reaction of N′-(2,4-dimethylphenyl)-2,2,2-trifluoroacetohydrazide with orthoformate and ammonium formate and direct cyclization to give 1-(2,4-dimethylphenyl)-3-(trifluoromethyl)-1H-1,2,4-triazole

23.2 g (0.1 mol) of N′-(2,4-dimethylphenyl)-2,2,2-trifluoroacetohydrazide are stirred together with 133 g (0.9 mol) of triethyl orthoformate and 19 g of ammonium formate for 6 hours at 80° C. The inorganic salts are filtered off and washed with a small amount of triethyl orthoformate. Distilling off the triethyl orthoformate gives 24.2 g of 96% strength crude product.

This can be further purified by recrystallization from a small amount of hexane.

NMR as under Example 3.

### Example 5b

### Reaction of N′-(2,4-dimethylphenyl)-2,2,2-trifluoroacetohydrazide with orthoformate and ammonium acetate and direct cyclization to give 1-(2,4-dimethylphenyl)-3-(trifluoromethyl)-1H-1,2,4-triazole

23.2 g (0.1 mol) of N′-(2,4-dimethylphenyl)-2,2,2-trifluoroacetohydrazide are stirred together with 133 g (0.9 mol) of triethyl orthoformate and 44 g of ammonium formate and 200 ml of acetonitrile for 6 hours at 85° C. The inorganic salts are filtered off and washed with a small amount of triethyl orthoformate. Distilling off the triethyl orthoformate and the acetonitrile gives 25 g of 91% strength crude product.

This can be further purified by recrystallization from a small amount of hexane.

H-NMR as in Example 3.

### Example 5c

### Reaction of N′-(2,4-dimethylphenyl)-2,2,2-trifluoroacetohydrazide with orthoformate and ammonia and direct cyclization to give 1-(2,4-dimethylphenyl)-3-(trifluoromethyl)-1H-1,2,4-triazole

23.2 g (0.1 mol) of N′-(2,4-dimethylphenyl)-2,2,2-trifluoroacetohydrazide are stirred together with 133 g (0.9 mol) of triethyl orthoformate and 30 g of ammonia and 200 ml of acetonitrile for 6 hours at 85° C. Distilling off the triethyl orthoformate and the acetonitrile gives 25 g of 79% strength crude product. The main by-product is uncyclized N′-(2,4-dimethylphenyl)-2,2-difluoroacetohydrazide. H-NMR as in Example 3.

### Example 6a

### Reaction of N′-(2,4-dimethylphenyl)-2,2-difluoroacetohydrazide with orthoformate and ammonium formate and direct cyclization to give 1-(2,4-dimethylphenyl)-3-(difluoromethyl)-1H-1,2,4-triazole

10 g (0.043 mol) of N′-(2,4-dimethylphenyl)-2,2-difluoroacetohydrazide are stirred together with 50 g (0.344 mol) of triethyl orthoformate and 13.5 g of ammonium formate for 24 hours at 80° C. The solvents are distilled off under reduced pressure and the residue is stirred with methylene chloride and water. The water phase is discarded and the organic phase is concentrated on a rotary evaporator. This gives 9.4 g of a 78% strength crude product. This can be further purified by recrystallization from cyclohexane.

H-NMR (CDCl3): 8.3 ppm (s, 1H); 7.1-7.25 (s+2d, 3H); 6.65-6.9 ppm (tr 1H); 2.4 ppm (s, 3H); 2.2 ppm (s, 3H)

### Reaction of 3-chloro-N′-(2,4-dimethylphenyl)-2,2,3,3-tetrafluoropropane hydrazide with orthoformate and ammonium formate and direct cyclization to give 3-(2-chloro-1,1,2,2-tetrafluoroethyl)-1-(2,4-dimethylphenyl)-1H-1,2,4-triazole

13.94 g of 90% strength 3-chloro-N′-(2,4-dimethylphenyl)-2,2,3,3-tetrafluoropropane hydrazide are stirred together with 50 g of triethyl orthoformate and 13.25 g of ammonium formate for 24 hours at 80° C. The solvents are distilled off under reduced pressure and the residue is stirred with methylene chloride and water. The water phase is discarded and the organic phase is concentrated on a rotary evaporator. Purification by column chromatography gives 6.6 g of 96.6% strength product (49% of theory).

### Example 6b

### Preparation of N′-(2,4-dimethylphenyl)-2,2-difluoroacetohydrazide

In 50 ml of ethanol, 8.9 g of methyl difluoroacetate are introduced as initial charge and, at RT, 10 g of 2,4-dimethylphenylhydrazine are added under a nitrogen atmosphere. The mixture is stirred overnight at room temperature and then the solvents are distilled off under reduced pressure. This gives 17 g of a 92% strength solid. This can be recrystallised from petroleum ether.

H-NMR (CDCl3): 8.1 ppm (s, 1H); 6.95 ppm (s+d, 2H); 6.7 ppm (d 1H), 5.9-6.15 ppm (tr 1H); 2.25-2.27 ppm (2 s, 6H)

### Example 7a

### Preparation of N′-(2,4-dimethylphenyl)-2,2,2-trifluoroacetohydrazide

In 50 ml of ethanol, 6.6 g of ethyl trifluoroacetate are introduced as initial charge and, at RT, 4 g of 2,4-dimethylphenylhydrazine are added under a nitrogen atmosphere. The mixture is stirred overnight at room temperature and then the solvents are distilled off under reduced pressure. This gives 5.8 g of a 90% strength product. This can be recrystallised from petroleum ether.

H-NMR (CDCl3): 8.16 ppm (br, 1H); 6.95-6.93 ppm (s+d, 2H); 6.67 ppm (d 1H), 6-4.5 ppm (sbr, 1H); 2.25 (s, 3H) 2.22 ppm (2 s, 6H)

### Example 7b

### Preparation of 3-chloro-N′-(2,4-dimethylphenyl)-2,2,3,3-tetrafluoropropane hydrazide

In 50 ml of ethanol, 12.6 g of methyl 3-chlorotetrafluoropropionate are added as initial charge and, at RT, 8 g of 2,4-dimethylphenylhydrazine are added under a nitrogen atmosphere. The mixture is stirred overnight at room temperature and then the solvents are distilled off under reduced pressure. This gives 11.5 g of a 98.9% strength product. This can be recrystallised from petroleum ether.

M+: 298

H¹-NMR (CDCl3): 8.22 ppm (br, 1H); 6.95-6.93 ppm (s+d, 2H); 6.68 ppm (d 1H), 6-5 ppm (sbr, 1H); 2.25 (s, 3H) 2.22 ppm (2 s, 6H).

1. A process for the preparation of a compound of formula (I) 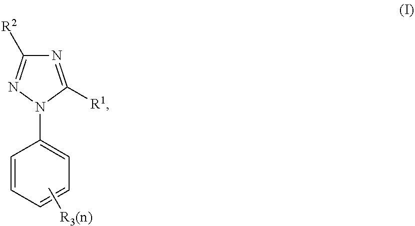
in which:
R¹ is H, alkyl, haloalkyl, aryl, heteroaryl, alkylaryl, or alkylheteroaryl,
R² is alkyl, haloalkyl, aryl, or heteroaryl,
R³ (n) is n number of identical or different radicals selected from the group consisting of hydrogen, (C₁-C₅)-alkyl, (C₁-C₅)-haloalkyl, (C₁-C₅)-alkylthio, (C₁-C₅)-alkyl-SO—, (C₁-C₅)-alkyl-SO₂—, (C₁-C₅)-haloalkylthio, (C₁-C₅)-haloalkyl-SO—, (C₁-C₅)-haloalkyl-SO₂—, —SO₂-halogen, —SO₂Cl, —SO₂H, —SH, (C₁-C₅)-alkyl-O—, (C₁-C₅)-haloalkyl-O—, halogen, hydroxy, nitro, amino, (C₁-C₅)-alkylamino, (C₁-C₅)-bisalkylamino, halogen-(C₁-C₅)-alkoxy, aryl, heteroaryl, (C₁-C₅)-alkylaryl, or (C₁-C₅)-alkylheteroaryl,
n is 1, 2, 3, 4, or 5,
wherein said process comprises converting
a compound of formula (II), 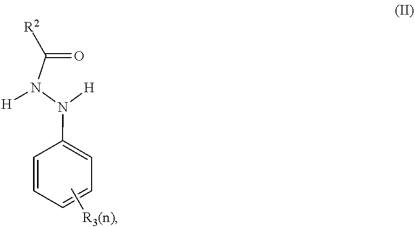
in which R², R³ and n are as defined above, to a compound of formula (III), 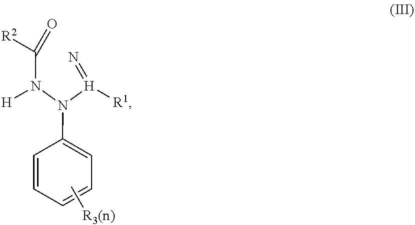
in which R¹, R², R³ and n are as defined above, which is cyclized with the elimination of water to give the compound of formula (I).

2. The process according to claim 1, comprising the preparation of the compound of formula (I) without interim isolation of the intermediate of the general formula (III).

3. The process according to claim 1, comprising isolating and converting the compound of formula (III) separately with the elimination of water to the compound of formula (I).

4. A compound of formula (III), 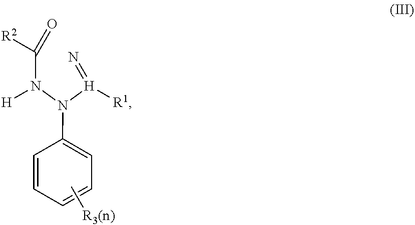
in which:
R¹ is H, alkyl, haloalkyl, aryl, heteroaryl, alkylaryl, or alkylheteroaryl,
R² is alkyl, haloalkyl, aryl, or heteroaryl,
R³ (n) is n number of identical or different radicals selected from the group consisting of hydrogen, (C₁-C₅)-alkyl, (C₁-C₅)-haloalkyl, (C₁-C₅)-alkylthio, (C₁-C₅)-alkyl-SO—, (C₁-C₅)-alkyl-SO₂—, (C₁-C₅)-haloalkylthio, (C₁-C₅)-haloalkyl-SO—, (C₁-C₅)-haloalkyl-SO₂—, —SO₂-halogen, —SO₂Cl, —SO₂H, —SH, (C₁-C₅)-alkyl-O—, (C₁-C₅)-haloalkyl-O—, halogen, hydroxy, nitro, amino, (C₁-C₅)-alkylamino, (C₁-C₅)-bisalkylamino, halogen-(C₁-C₅)-alkoxy, aryl, heteroaryl, (C₁-C₅)-alkylaryl, or (C₁-C₅)-alkylheteroaryl,
n is 1, 2, 3, 4, or 5.

5. The process according to claim 1, wherein the compound of formula (II) is converted to the compound of formula (III) by reacting with a compound of formula (IV), 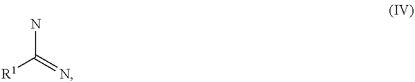
wherein R¹ is as defined in claim 1.

6. The process according to claim 1, wherein the compound of formula (II) is reacted with an ammonium salt of formula (VI)
NH₄X (VI),
in the presence of a formic acid derivative of formula (V), 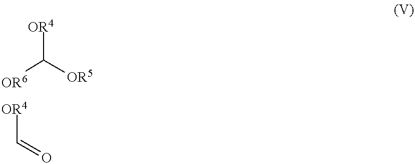
in which:
R⁴ is hydrogen, (C₁-C₅)-alkyl, (C₁-C₅)-haloalkyl, aryl, heteroaryl, (C₁-C₅)-alkylaryl, or (C₁-C₅)-alkylheteroaryl,
R⁵ is hydrogen, (C₁-C₅)-alkyl, (C₁-C₅)-haloalkyl, aryl, heteroaryl, (C₁-C₅)-alkylaryl, or (C₁-C₅)-alkylheteroaryl,
R⁶ is hydrogen, (C₁-C₅)-alkyl, (C₁-C₅)-haloalkyl, aryl, heteroaryl, (C₁-C₅)-alkylaryl, or (C₁-C₅)-alkylheteroaryl,
X is an organic or inorganic acid radical R⁷COO—, Hal-, HSO₄, — or SO₄—,
and R² is hydrogen, (C₁-C₅)-alkyl, (C₁-C₅)-haloalkyl, aryl, heteroaryl, (C₁-C₅)-alkylaryl, or (C₁-C₅)-alkylheteroaryl,
to give the compound of formula (III), 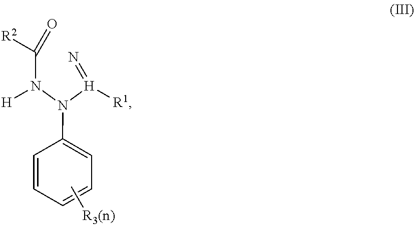
in which:
R¹ is hydrogen,
R², R³ and n are as defined in claim 1.

7. The process according to claim 1, wherein the compound of general formula (II) is reacted with ammonia in the presence of a formic acid derivative of formula (V), 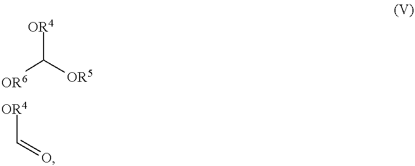
in which:
R⁴ is hydrogen, (C₁-C₅)-alkyl, (C₁-C₅)-haloalkyl, aryl, hetero aryl, (C₁-C₅)-alkylaryl or, (C₁-C₅)-alkylheteroaryl,
R⁵ is hydrogen, (C₁-C₅)-alkyl, (C₁-C₅)-haloalkyl, aryl, heteroaryl, (C₁-C₅)-alkylaryl, or (C₁-C₅)-alkylheteroaryl,
R⁶ is hydrogen, (C₁-C₅)-alkyl, (C₁-C₅)-haloalkyl, aryl, heteroaryl, (C₁-C₅)-alkylaryl, or (C₁-C₅)-alkylheteroaryl,
to give the compound of formula (III), 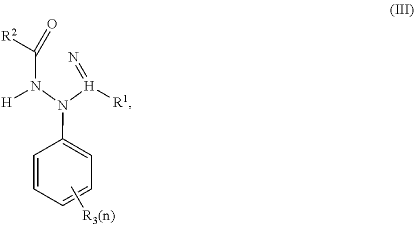
in which:
R¹ is hydrogen,
R², R³ and n are as defined in claim 1.
